(19) United States
(12) Patent Application Publication
(10) Pub. No.: US 2024/0138871 A1
(43) Pub. Date: May 2, 2024

(54) THICK TISSUE APPLICATION OF DIRECTIONAL ENERGY TO JAW MEMBERS

(71) Applicant: Covidien LP, Mansfield, MA (US)

(72) Inventors: Daniel A. Joseph, Golden, CO (US); Kelsey S. Fitzgerald, Superior, CO (US); Jennifer R. Mchenry, Denver, CO (US); Robert F. McCullough, JR., Boulder, CO (US)

(21) Appl. No.: 17/976,011

(22) Filed: Oct. 28, 2022

(51) Int. Cl. A61B 17/295; A61B 18/14

(52) U.S. Cl. CPC A61B 17/295; A61B 18/1445; A61B 2018/0063; A61B 2018/1455

(57) ABSTRACT

A surgical instrument includes a housing having a shaft extending distally from the housing and an end effector disposed at a distal end of the shaft with first and second jaw members. The jaw members are moveable relative to one another from a first, open configuration spaced relative to one another, to a second, closed configuration. The first jaw member includes a sealing plate having proximal and distal segments each adapted to connect to an energy source via a single, wired connection and the second jaw member includes a sealing plate adapted to connect to the energy source. A resistor is disposed in circuit and is electrically coupled to the energy source. The resistor is configured to regulate the flow of energy to the proximal segment of the first jaw member via the single wired connection upon activation and until the jaw members reach the second configuration.

### BACKGROUND

### 1. Technical Field

The present disclosure relates to electrosurgical forceps and more particularly, to a thick tissue sensor to direct the application of energy for use with an endoscopic or open bipolar and/or monopolar electrosurgical forceps for sealing, cutting, and/or coagulating tissue.

### 2. Discussion of Related Art

Electrosurgical forceps utilize both mechanical clamping action and electrical energy to affect hemostasis by heating the tissue and blood vessels to coagulate, cauterize and/or seal tissue. Electrosurgical forceps may be open forceps for use during open surgical procedures or may be endoscopic or laparoscopic forceps for remotely accessing organs through smaller, puncture-like incisions.

Many surgical procedures require cutting or ligating blood vessels or vascular tissue. By utilizing an endoscopic electrosurgical forceps, a surgeon can cauterize, coagulate/desiccate, and/or simply reduce or slow bleeding simply by controlling the intensity, frequency and duration of the electrosurgical energy applied through the jaw members to the tissue.

In order to effectively seal vessels (or tissue) two predominant mechanical parameters must be accurately controlled—the pressure applied to the vessel (tissue) and the gap distance between the electrodes-both of which are affected by the thickness of the tissue. The pressure applied to the vessel may be fixed by the mechanical design of the instrument.

It can be difficult for surgeons to visually determine the gap distance between electrodes before energy application. After energy application, it may be difficult to ensure that the jaws of the forceps have achieved an appropriate seal closure. The visualization of the surgical field may be difficult because of blood within the surgical field, lack of complete vessel (tissue) dissection, or isolation.

### SUMMARY

Provided in accordance with the present disclosure is a surgical instrument which includes a housing having a shaft extending distally from the housing and an end effector disposed at a distal end of the shaft with first and second jaw members. The jaw members are moveable relative to one another from a first, open configuration spaced relative to one another, to a second, closed configuration. The first jaw member includes a sealing plate having proximal and distal segments each adapted to connect to an energy source via a single, wired connection and the second jaw member includes a sealing plate adapted to connect to the energy source. A resistor is disposed in circuit and is electrically coupled to the energy source. The resistor is configured to regulate the flow of energy to the proximal segment of the first jaw member via the single wired connection upon activation and until the jaw members reach the second configuration.

In aspects in accordance with the present disclosure, the surgical instrument further includes a sensor disposed on the jaw members and configured to determine the size of a gap distance between jaw members to determine when the jaw members are disposed in the second, closed configuration. In other aspects in accordance with the present disclosure, the sensor is disposed on the jaw members and relays data to the energy source to regulate the energy to the proximal segment of the first jaw member.

In aspects in accordance with the present disclosure, one or more switches are configured in circuit with the single wired connection and the resistor to regulate the flow of energy to the proximal segment. In other aspects in accordance with the present disclosure, the resistor is a variable resistor.

In aspects in accordance with the present disclosure, activation of the resistor is controlled by an algorithm associated with the energy source. In other aspects in accordance with the present disclosure, activation of the resistor is controlled by mechanical movement of the jaw members or the shaft.

Provided in accordance with the present disclosure is a surgical instrument which includes a housing having a shaft extending distally from the housing and an end effector disposed at a distal end of the shaft with first and second jaw members. The first and second jaw members are moveable relative to one another from a first, open configuration wherein the first and second jaw members are spaced relative to one another, to a second, closed configuration wherein the first and second jaw members are closer to one another for approximating tissue. The first and second jaw members are configured to define a gap distance therebetween for receiving tissue. The first jaw member includes a sealing plate having proximal and distal segments each adapted to connect to an energy source via a single wired connection, the second jaw member includes a sealing plate and is adapted to connect to the energy source. A sensor is disposed on the jaw members and is configured to determine the size of the gap distance and relay data relating to the gap distance to the energy source. A resistor is disposed in circuit and is electrically coupled to one or more switches and is configured to regulate the flow of energy to the proximal segment of the first jaw member based on the data.

In aspects in accordance with the present disclosure, the sensor is a proximity sensor, a first portion of the proximity sensor disposed on the first jaw member and a second portion of the proximity sensor disposed on the second jaw member.

In aspects in accordance with the present disclosure, the sensor relays data to the energy source to regulate the energy to the proximal segment of the first jaw member.

In aspects in accordance with the present disclosure, the one or more switches are in circuit with the single wired connection and the resistor to regulate the flow of energy to the proximal segment. In aspects in accordance with the present disclosure, the resistor is a variable resistor.

In aspects in accordance with the present disclosure, activation of the resistor is controlled by an algorithm associated with the energy source. In aspects in accordance with the present disclosure, activation of the resistor is controlled by mechanical movement of movement of the jaw members or the shaft.

In aspects in accordance with the present disclosure, the second jaw member includes proximal and distal segments each adapted to connected to an energy source via a single wired connection. In other aspects in accordance with the present disclosure, a second resistor is disposed in circuit and electrically coupled to the energy source, the second resistor is configured to regulate the flow of energy to the proximal segment of the second jaw member via the single wired connection upon activation and until the jaw members reach the second configuration. In yet other aspects in accordance with the present disclosure, the first and second resistors cooperate to regulate the flow of energy to the proximal segments of the first and second jaw members, respectively.

### BRIEF DESCRIPTION OF THE DRAWINGS

Various aspects of the present disclosure are described hereinbelow with reference to the drawings, which are incorporated in and constitute a part of this specification, wherein:

FIG. 1 is a side view of an endoscopic electrosurgical forceps in accordance with the present disclosure;

FIG. 2 is an internal, perspective, cut-away view of the endoscopic electrosurgical forceps of FIG. 1 showing a handle and drive assembly in an initial position;

FIG. 3 is an enlarged, front perspective view an end effector assembly of the forceps of FIG. 1 shown in an open configuration with tissue “T” disposed between a pair of first and second jaw members;

FIGS. 4A and 4B are schematic diagrams showing a resistor and generator circuit diagram for use in accordance with the forceps of FIG. 1; and

FIG. 5 is a schematic view of a robotic surgical system in accordance with the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closest to the clinician and the term “distal” refers to the portion of the device or component thereof that is farthest from the clinician.

This disclosure generally includes one or more sensors positioned in or on the body of an electrosurgical forceps to determine the thickness of the tissue grasped between the jaw members prior to activation of the jaw members which, in turn, activates an electrical algorithm to selectively regulate the resistance of the proximal end the jaw member upon initial activation of the jaw members to direct more heat to the distal end of the jaw members and more evenly treat the tissue during sealing.

Any type of sensor may be utilized for this purpose to sense a thick tissue condition, e.g., a position sensor disposed on the closure tube or activation rod, a sensor disposed on the cam slot, a sensor that determiners the relative position or angle of a flag adjacent a distal end of the jaw member, etc. The sensor may provide feedback (e.g., audible, tactile, or visual) to a generator (or a clinician) for adjusting the electrosurgical parameters of the electrosurgical energy during a sealing cycle based on the tissue thickness.

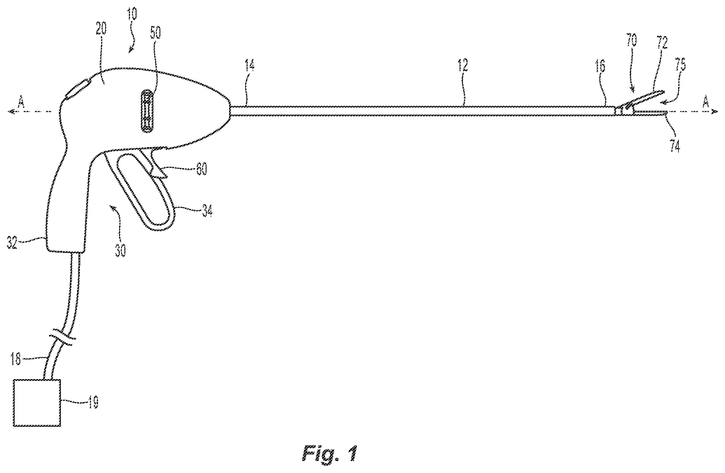

Referring now to FIG. 1, a laparoscopic electrosurgical forceps 10 is provided in accordance with the present disclosure and includes a housing 20, a handle assembly 30, a rotating assembly 50, a trigger assembly 60, and an end effector assembly 70. For the purposes herein, an endoscopic forceps may also be utilized. The end effector assembly 70 includes first and second jaw members 72, 74 for grasping, sealing, and treating tubular vessels and vascular tissue. For the purposes herein, forceps 10 will be described generally.

The forceps 10 includes a shaft 12 that defines a longitudinal axis “A-A” of the forceps 10 and has a proximal end 14 and a distal end 16. The proximal end 14 of the shaft 12 is operatively engaged to the housing 20. The distal end 16 of the shaft 12 is configured to mechanically engage the end effector assembly 70 to move the first and second jaw members 72, 74 between an open configuration and a closed configuration for grasping tissue. As discussed herein, one or more various mechanical or electrical components may be utilized to determine closure of the first and second jaw members 72, 74 relative to one another depending upon a particular purpose. This becomes relevant when sealing thick tissue. For example, tissue sensors, proximity sensors, angular alignment sensors, tissue impedance monitors, component alignment or registry sensors, etc. have all been utilized to mechanically and electrically define when jaw members are “open”, “spaced”, or sufficiently “closed” for particular purposes. Various algorithms, adjustments and/or energy compensations have been proposed for particular instances for various tissue treatments to compensate for tissue sealing when the jaw members have been nonparallel, angled or sufficiently closed. As used herein, the term “closed configuration” may be defined as one of these terms and may not need be fully closed but closed enough to be suited for a particular surgical purpose.

Forceps 10 also includes an electrosurgical cable 18 that connects the forceps 10 to a source of electrosurgical energy, e.g., a generator 19. The source of electrosurgical energy 19 provides electrosurgical energy to the end effector 70 of the forceps 10. It is also contemplated that the forceps 10 may include an energy source, e.g., a battery (not shown) positioned on or within the housing 20 to provide electrosurgical energy to the forceps 10.

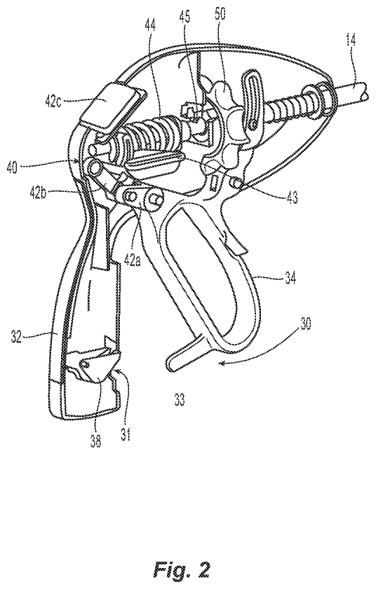

Referring to FIG. 2, the handle assembly 30 includes a fixed handle 32 and a moveable handle 34. The fixed handle 32 is integrally associated with housing 20 and the moveable handle 34 is movable relative to the fixed handle 32 to actuate a drive assembly 40 as described in detail below. The rotating assembly 50 is disposed substantially within the housing 20 and is rotatable approximately 180 degrees in either direction about the longitudinal axis “A-A” to rotate the end effector assembly 70 relative to the housing 20.

A flange 33 extends from a proximal end of the handle 34 and is configured to engage an aperture 31 defined within handle 32 and ultimately engage a latch 38 that is configured to selectively lock and unlock the handles 32 and 34 relative to one another as needed during surgery. Upon initial movement and engagement of the flange 33 into aperture 31 with latch 38 the handles 32, 34 will lock with one another and upon subsequent movement relative to one another, the handles 32, 34 would unlock.

Briefly describing operation of the drive assembly 40, the moveable handle 34 operably couples to a series of links 42a-42c that cooperate to move a carriage 43 proximally against a compression spring 44 which, in turn, regulates movement of a drive tube 45 that translates through shaft 12 and regulates the overall closure force of jaw member 72 relative to jaw member 70. More particularly, the translation the drive tube 45 controls the proximal movement of a cam pin 95 within cam slots 76 and 78 defined within respective jaw members 72 and 74, which, in turn, forces jaw member 72 to move about pivot 76 relative to jaw member 74 when pushed distally to grasp tissue “T” disposed within a space 75 between the jaw members 72, 74.

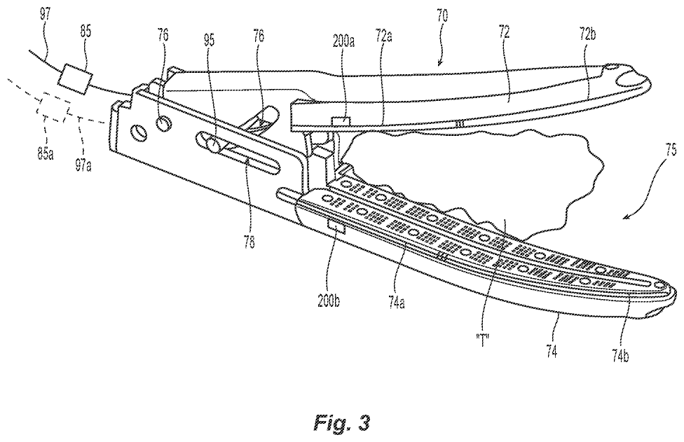

As mentioned above by way of example in FIG. 3, sensors 200a, 200b may be disposed on jaw members 72, 74, respectively, and provide feedback to generator 19 (or a clinician) regarding the thickness of the tissue “T” disposed between the jaw members 72, 74. Other types of sensors are also contemplated and additional sensors may be employed on other parts of the jaw members, e.g., proximal or distal segment of jaw member 72, e.g., 72a, 72b respectively, a proximal or distal segment of jaw member 74, e.g., 74a, 74b respectively, or any other part of the forceps 10 such as shaft 12, drive tube 45 or housing 20.

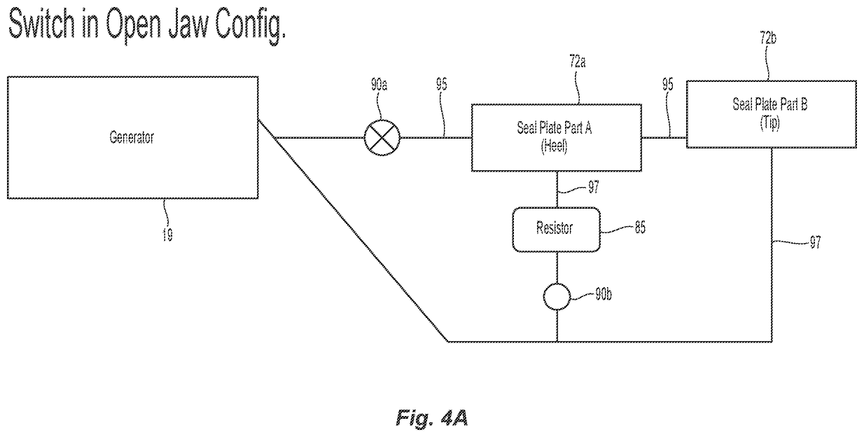

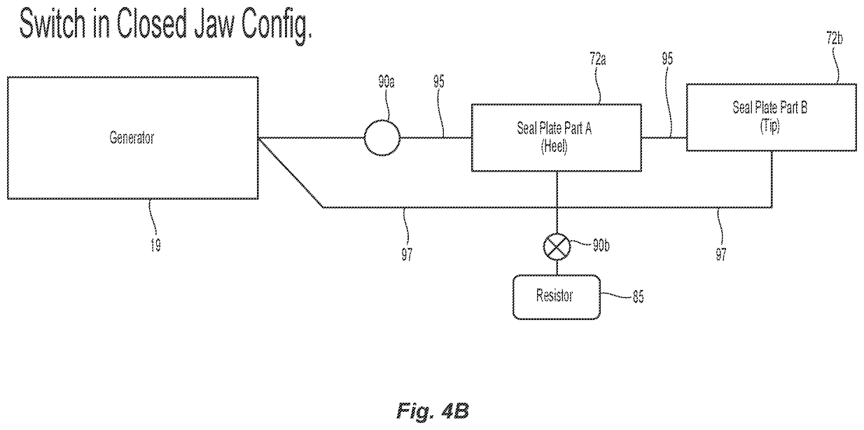

Turning now to FIGS. 4A and 4B which show a schematic representation of the jaw member of FIG. 3, with FIG. 4A indicating a jaw “open” configuration with thick tissue being disposed between the jaw members 72, 74 when the sensors 200a, 200b read a thick tissue condition and FIG. 4B indicating the jaw members being in a closed configuration as detected by sensors 200a, 200b. More particularly, and turning initially with respect to FIG. 4A, when thick tissue is sensed between jaw members 72, 74, sensors 200a, 200b send a signal to the generator 19 (as discussed above) and an algorithm is initiated (or a clinician may be alerted to initiate an algorithm). This regulates the resistance of the proximal segment 72a of the jaw member 72 relative to the distal segment 72b of the jaw member 72 in order to provide more energy during the initial sealing process to the distal end of the jaw members 72, 74 to facilitate a better and higher quality seal.

In embodiments, the resistance of the proximal segment 74a of jaw member 74 relative to the distal segment 74b the jaw member 74 may also be regulated in combination with (or in the alternative) to provide more energy during the initial sealing process to the distal end of the jaw members 72, 74 to facilitate a better and higher quality seal.

It is contemplated that unlike traditional multi-segmented seal-plated jaw members wherein each seal plate segment must be separately wired and multiplexed back to the generator, in this instance, the seal plate end, namely, respective proximal and distal segments 72a, 74a, and 74a, 74b, may share the same common wires 95, 97 therebetween. When disposed in an open configuration as depicted in FIG. 4A, the sensors 200a, 200b relay data back to the generator 19 to close a switch 90a disposed within the connection path, e.g., wire 95 and open switch 90b disposed within connection path, e.g., wire 97. During activation, energy is regulated by one or more resistors 85 more towards the distal end of the jaw members 72, 74 based on an algorithm. The one or more resistors 85 may be electrically introduced or mechanically introduced into the circuit. Resistor 85 may be any kind of resistor, e.g., fixed or variable.

It is important to note that switch 90a may remain open (or not even be included, e.g., only the resistor 85 is in the circuit) and the active resistance of resistor 85 may in and of itself work to offset the energy difference necessary to allow the distal tip to effectively treat tissue initially prior to equalization and eventual tissue seal.

In embodiments, one or both jaw members 72, 74 may be segmented depending upon a particular purpose. In other embodiments, the algorithm may be regulated based on the thickness of the tissue “T” as estimated by the sensed gap between the jaw members 72, 74 by sensors 200a, 200b. In still other embodiments, the energy profile between the distal and proximal ends, e.g., proximal segment 72a and distal segment 72b of jaw member 72, may equalize or even reverse as the seal cycle nears completion. The one or more resistors 85 may be electrically or mechanically eliminated from the circuit by a step in the algorithm or by mechanical movement, e.g., movement of the jaw members 72, 74 relative to one another (e.g., the act of closing the jaw members, cuts off the resistor 85 from the circuit like a brush contact or spring contact).

Turning now to FIG. 4B, as the seal cycle begins and the thick tissue at the distal segments 72a, 74a of the jaw members 72, 74 begins to shrink (the tissue begins to seal), the jaw members 72, 74 further approximate towards a more parallel closure which is known to be more conducive to a better seal, a more traditional sealing algorithm and energy delivery profile may be utilized. As such, the one or more resistors 85 may be taken off-line or out of the energy delivery circuit. Obviously, stopping the tissue sealing process at this point to take a component offline is not practical. In this instance, the sensors 200a, 200b may be configured to sense the distance between the jaw members 72, 74 (or the angle therebetween as mentioned above) and a signal may be sent to the generator 19 to close a switch 90b disposed within the connection path, e.g., wire 97, and open a switch 90a disposed within connection path, e.g., wire 95. Energy from the generator 19 is then regulated to seal tissue as intended to jaw members 72, 74 based on the same or a new algorithm without going through the resistor 85. The one or more resistors 85 may be electrically taken offline or mechanically taken offline by movement of the jaw members 72, 74 (or another part of the forceps 10).

It is important to note that the forceps 10 may be designed as simple as using one energy delivery sealing algorithm with just one ON/OFF line resistor 85 as described above to a forceps with many levels of different energy regulating algorithms based on sensor 200a, 200b input prior to and during sealing as it concerns the thickness of the tissue and the recommended energy profile from distal to proximal end, etc.

In embodiments, jaw member 74 may also be configured to include a resistor depending upon a particular purpose, e.g., resistor 85a (shown in phantom) and connect to a proximal end 74b of jaw member 74 via wire 97a (see FIG. 3). Resistor 85a works in a similar fashion as described above with respect to resistor 85. Generator 19 may also be configured to control resistor 85a in combination with or separately from resistor 85.

In embodiments, the jaw members 72, 74 and resistors may be wired in a different fashion (not shown) wherein energy to the distal end of one or both jaw members is regulated to the proximal end. In other words, it is contemplated that directional energy may be regulated proximally and distally depending upon a particular purpose.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

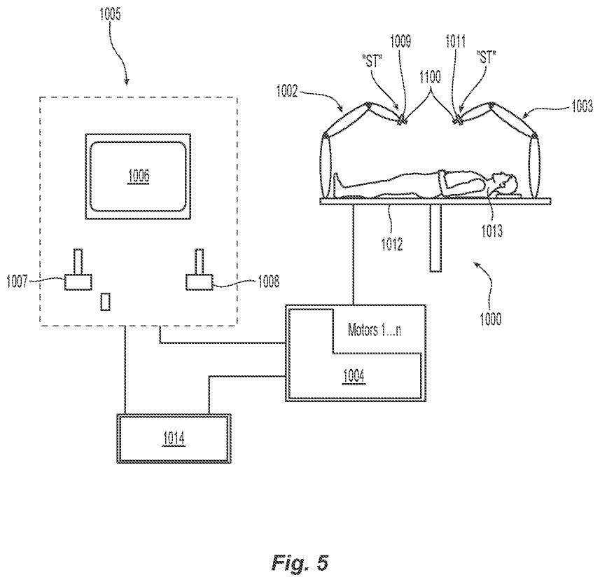

Referring initially to FIG. 5, a medical workstation is shown generally as workstation 1000 and generally may include a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100, in accordance with any one of several embodiments disclosed herein, as will be described in greater detail below.

Robot arms 1002, 1003 may be driven by electric drives (not shown) that are connected to control device 1004. Control device 1004 (e.g., a computer) may be set up to activate the drives, in particular by means of a computer program, in such a way that robot arms 1002, 1003, their attaching devices 1009, 1011 and thus the surgical tool (including end effector 1100) execute a desired movement according to a movement defined by means of manual input devices 1007, 1008. Control device 1004 may also be set up in such a way that it regulates the movement of robot arms 1002, 1003 and/or of the drives.

Medical workstation 1000 may be configured for use on a patient 1013 lying on a patient table 1012 to be treated in a minimally invasive manner by means of end effector 1100. Medical workstation 1000 may also include more than two robot arms 1002, 1003, the additional robot arms likewise being connected to control device 1004 and being telemanipulatable by means of operating console 1005. A medical instrument or surgical tool (including an end effector 1100) may also be attached to the additional robot arm. Medical workstation 1000 may include a database 1014, in particular coupled to with control device 1004, in which are stored, for example, pre-operative data from patient/living being 1013 and/or anatomical atlases.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A surgical instrument, comprising:
a housing;
a shaft extending distally from the housing;
an end effector disposed at a distal end of the shaft and including first and second jaw members moveable relative to one another from a first, open configuration wherein the first and second jaw members are spaced relative to one another, to a second, closed configuration wherein the first and second jaw members are mechanically or electrically defined as closed by a switch or algorithm, the first jaw member including a sealing plate having proximal and distal segments each adapted to connect to an energy source via a single, wired connection, the second jaw member including a sealing plate and adapted to connect to the energy source; and
a resistor disposed in circuit and electrically coupled to the energy source, the resistor configured to regulate the flow of energy to the proximal segment of the first jaw member via the single wired connection upon activation and until the jaw members reach the second configuration.

2. The surgical instrument according to claim 1, further comprising a sensor disposed on the jaw members and configured to determine the size of a gap distance between jaw members to determine when the jaw members are disposed in the second, closed configuration.

3. The surgical instrument according to claim 2, wherein the sensor is disposed on the jaw members and relays data to the energy source to regulate the energy to the proximal segment of the first jaw member.

4. The surgical instrument according to claim 1, further comprising one or more switches configured in circuit with the single wired connection and the resistor to regulate the flow of energy to the proximal segment.

5. The surgical instrument according to claim 1, wherein the resistor is a variable resistor.

6. The surgical instrument according to claim 1, wherein activation of the resistor is controlled by an algorithm associated with the energy source.

7. The surgical instrument according to claim 1, wherein activation of the resistor is controlled by at least one of movement of the jaw members or the shaft.

8. A surgical instrument, comprising:
a housing;
a shaft extending distally from the housing;
an end effector disposed at a distal end of the shaft and including first and second jaw members moveable relative to one another from a first, open configuration wherein the first and second jaw members are spaced relative to one another, to a second, closed configuration wherein the first and second jaw members are closer to one another for approximating tissue, the first and second jaw members defining a gap distance therebetween for receiving tissue, the first jaw member including a sealing plate having proximal and distal segments each adapted to connect to an energy source via a single wired connection, the second jaw member including a sealing plate and adapted to connect to the energy source;
a sensor disposed on the jaw members and configured to determine the size of the gap distance and relay data relating to the gap distance to the energy source; and
a resistor disposed in circuit and electrically coupled to one or more switches and configured to regulate the flow of energy to the proximal segment of the first jaw member based on the data.

9. The surgical instrument according to claim 8, wherein the sensor is a proximity sensor, a first portion of the proximity sensor disposed on the first jaw member and a second portion of the proximity sensor disposed on the second jaw member.

10. The surgical instrument according to claim 8, wherein the sensor relays data to the energy source to regulate the energy to the proximal segment of the first jaw member.

11. The surgical instrument according to claim 8, wherein the one or more switches are in circuit with the single wired connection and the resistor to regulate the flow of energy to the proximal segment.

12. The surgical instrument according to claim 8, wherein the resistor is a variable resistor.

13. The surgical instrument according to claim 8, wherein activation of the resistor is controlled by an algorithm associated with the energy source.

14. The surgical instrument according to claim 8, wherein activation of the resistor is controlled by mechanical movement of at least one of movement of the jaw members or the shaft.

15. The surgical instrument according to claim 8, wherein the second jaw member includes proximal and distal segments each adapted to connected to an energy source via a single wired connection.

16. The surgical instrument according to claim 15, wherein a second resistor is disposed in circuit and electrically coupled to the energy source, the second resistor is configured to regulate the flow of energy to the proximal segment of the second jaw member via the single wired connection upon activation and until the jaw members reach the second configuration.

17. The surgical instrument according to claim 16, wherein the first and second resistors cooperate to regulate the flow of energy to the proximal segments of the first and second jaw members, respectively.
